(19) United States
(12) Patent Application Publication
(10) Pub. No.: US 2008/0226713 A1
(43) Pub. Date: Sep. 18, 2008

(54) Controlled Release Pharmaceutical Compositions of Tolperisone for Oral Administration

(76) Inventors: Angelika Bodenteich, Wien (AT); Eberhard Pirich, Wien (AT); Josef Bockmann, Modling (AT); Werner Frantsits, Wien (AT)

Correspondence Address: Roberta L. Robins;Robins & Pasternak, 1731 Embarcadero Road, Suite 230, Palo Alto, CA, 94303, US

(21) Appl. No.: 10/591,004

(22) PCT Filed: Mar. 7, 2005
(86) PCT No.: PCT/EP2005/002379

§ 371 Date: Oct. 29, 2007

(30) Foreign Application Priority Data
Mar. 5, 2004 (AT) A 386/2004

(51) Int. Cl. A61K 9/22; A61K 31/445

(52) U.S. Cl. 424/468; 514/317

(57) ABSTRACT

The invention relates to a tolperisone-containing pharmaceutical preparation with controllable active-substance release for oral administration, characterised in that the active substance tolperisone and/or a pharmaceutical salt thereof is embedded in a pharmaceutically compatible material. By selecting the pharmaceutically compatible materials in the preparation and accordingly in the coating of a tablet or granule, a specific release of active substance can be adjusted which is matched to the special genotype in the metabolisation of tolperisone. At the same time, as a result of the very uniform and persistent release of tolperisone, the in vivo inversion of enantiomerically pure tolperisone that is known from the art can be adjusted in favour of the R(−)tolperisone which is prominent in muscle-relaxing therapy.

### FIELD OF THE INVENTION

The present invention relates to a tolperisone-containing pharmaceutical preparation with controllable active-substance release for oral administration.

### BACKGROUND OF THE INVENTION

Tolperisone is the international non-proprietary name for the muscle relaxant (RS)-2,4′dimethyl-3-piperidinopropiophenone. The enantiomeric separation of tolperisone present as racemate is described in JP-A-53-40779. In this case, enantiomerically pure tolperisone is formed by diastereomer formation from racemic tolperisone and enantiomerically pure acetylphenylglycine salts. The diastereomers were separated by selective precipitation so that after separation of the acetylphenylglycine groups both R(−) and S(+)tolperisone was obtained in enantiomerically pure form.

Zsila et al. (Chirality 12: 720-726, 2000) have also dealt with the stereochemistry of tolperisone and established that the absolute configuration of (−)tolperisone corresponds to an R-configuration. This has also been confirmed by a monocrystal analysis which has shown that (+)tolperisone corresponds to the S configuration.

The pharmacological effect of the two enantiomers was also discussed in JP-A-53-40779. The pharmacological investigations describe a muscle-relaxing effect of R-tolperisone and a vaso- or bronchodilatory effect of S-tolperisone.

Despite the proven pharmaceutical efficacy of enantiomerically pure tolperisone and its pharmaceutically compatible salts, the oral administration is problematical insofar as the desired effect diminishes rapidly and the patient must therefore take tolperisone-containing preparations several times a day whereby the gastrointestinal tract of the patient can sometimes be damaged.

Tolperisone is metabolised relatively rapidly in the body wherein the enzyme CYP2D6 substantially influences the type and duration of the metabolism. Four different genotypes have been determined for this enzyme, namely “poor metabolisers” (approximately 7% of the population), “ultrafast metabolisers” (approximately 3%), “extensive metabolisers” (approximately 45%) and “intermediate metabolisers” (approximately 45%). The last two groups mentioned are only genotypically distinguishable but not phenotypically distinguishable. Especially in the group of “poor metabolisers”, there is a risk of toxicity since tolperisone is only converted very slowly.

In order to nevertheless achieve the desired long-term effect, it was proposed in JP-A-3277239 to develop transdermal formulations. However, practice shows that transdermal transport of medicinal products is limited especially with regard to dosage since unit doses of max. 150 mg can only be administered transdermally whereby an effective therapy is not yet established.

WO-A-00/59508 describes tolperisone-containing formulations which can be administered orally but do not have the disadvantages of the known tolperisone preparations which can be administered orally. In this case, an attempt was made to utilise the delayed effect of tolperisone insofar as the release behaviour of tolperisone should also be influenced by a defined selection of the enantiomeric ratio of R(−) to S(+)tolperisone. The adjustment of a defined enantiomeric ratio by chemical reaction is occasionally expensive and besides need not result in the desired pharmaceutical effect. Thus, in their article “Determination of Tolperisone Enantiomers in Plasma and Their Disposition in Rats” (Chem. Pharm. Bull. 4001, 272-274, Vol. 40 (1992)), Teruyoshi Yokoyama et al. have shown that an in-vivo inversion can be detected when using enantiomerically pure tolperisone. This means that through this in-vivo inversion enantiomerically pure S(+)tolperisone is converted into R(−)tolperisone to an extent of up to 20% or enantiomerically pure R(−)tolperisone is converted into S(+)tolperisone in a fraction up to 20%. This in-vivo inversion can reduce the desired pharmaceutical effect and also casts into question the use of enantiomerically pure tolperisone.

The object of the invention is to influence this in-vivo inversion by a particular, orally administrable pharmaceutical formulation wherein at the same time the controllability of the active substance release should also be modulated with the objective of long-term therapy.

### DESCRIPTION OF THE INVENTION

According to the present invention, a controlled release pharmaceutical composition for oral administration of tolperisone to a subject contains an amount of enantiomeric mixture of tolperisone, or pharmaceutically acceptable salts thereof, and a controlled release agent to provide for controlled release of the enantiomeric mixture of tolperisone upon such oral administration resulting in stereoselective disposition of tolperisone enantiomers in the blood plasma of the subject wherein the plasma area under the curve (AUC) concentration ratio of R-tolperisone to S-tolperisone is higher than that of a non-controlled release composition containing the same amount of enantiomeric mixture of tolperisone. Alternatively, the pharmaceutical composition may further contain (a) a core which includes (i) the enantiomeric mixture of tolperisone and (ii) the controlled release agent and (b) a controlled release coating associated with the core. By definition, an “enantiomeric mixture” of tolperisone contains both enantiomers R and S in more than trace amounts, i.e. with each at least 2% by weight. This is illustrated by a variety of mixtures, such as without limitation 10% S and 90% R tolperisone, a mixture of 98% R and 2% S, or a racemic mixture. Also by definition, a “racemic mixture” of tolperisone, or racemic tolperisone, has equal or almost equal amounts of the R and S enantiomers, meaning both enantiomers are present with each at least 45% by weight. This is illustrated by a mixture of 45% R and 55% S tolperisone. In the preferred embodiment, the enantiomeric mixture of tolperisone in the core is a racemic mixture, and the amount of racemic tolperisone in the core is within the range of 100-500 mg. As an alternative embodiment, the enantiomeric mixture of tolperisone may have at least 50% by weight the R-tolperisone and no less than 10% by weight the S-tolperisone.

The controlled release agent may be a mixture of anionic and cationic polymers, which may be exemplified by a mixture of Eudragit RS, Eudragit L and Eudragit S. The controlled release coating may be pH independent, i.e. meaning that the acidic or basic pH of the gastrointestinal tract do not appreciably effect dissolution of the active drug. Alternatively, the coating may be pH dependent, especially where it is resistant to acidic environment, favoring dissolution post-gastricly.

The subject receiving oral administration may be any mammal, preferentially a human.

By definition, “controlled release” involves dissolution profiles like those of examples 1-8, but excludes the dissolution profile of example 9 which exemplified “non-controlled release”. In general, “controlled release” results in no more than 80% (by weight) release at two hours (measured using the USP Basket Method at 75 rpm in 1,000 ml 0.1 N HCL at 37° C. “Non-controlled” release encompasses the range of more than 80% (by weight) release at one hour. According to preferred embodiments, such cut-off for controlled release of 100-249 mg of tolperisone may be no more than 45% or 55% (by weight) release at 2 hours. Also, such cut-off for controlled release of 250-500 mg of tolperisone may be no more than 20% or 30% by weight release at 2 hours.

Also according to the present invention, controlled release pharmaceutical composition for oral administration of tolperisone to a subject contains an amount of racemic tolperisone, or pharmaceutically acceptable salts thereof, and a controlled release agent to provide for controlled release of the racemic tolperisone upon such oral administration resulting in stereoselective disposition of tolperisone enantiomers in the blood plasma of the subject wherein the plasma area under the curve (AUC) concentration ratio of R-tolperisone to S-tolperisone is 3:1 or higher. In the preferred embodiment, the plasma area under the curve (AUC) concentration ratio is 4:1 or higher, and the amount of racemic tolperisone in the core is within the range of 100-500 mg. As an alternative, the pharmaceutical composition may further contain (a) a core which includes (i) the racemic tolperisone and (ii) the controlled release agent and (b) a controlled release coating associated with the core.

Further according to the present invention, a method of oral administration of tolperisone to a subject involves, oral administration by controlled release of a dose of an amount of racemic tolperisone in the range of 100-500 mg to provide a stereoselective disposition of tolperisone enantiomers in the blood plasma of the subject. In a preferred range of 250-500 mg of racemic tolperisone, wherein the plasma area under the curve (AUC) concentration of R-tolperisone is 100 ng*h/ml or higher and such concentration of S-tolperisone is 25 ng*h/ml or lower. In the preferred embodiment, the amount of racemic tolperisone is in the range of about 300 mg.

Throughout this specification and attached claims, “about” when modifying a single number such as “about 50” means the number ±10% such as 50±10%. When modifying a range such as “about 50-100”, it means the range consisting of the lower number −10% and the higher number +10% such as 45-110.

As further examples of preferred embodiments, the controlled release pharmaceutical composition may have racemic tolperisone in the amount of 100-200 mg, or pharmaceutically acceptable salts thereof, wherein the composition exhibits an in vitro dissolution profile (measured using the USP Basket Method at 75 rpm in 1,000 ml 0.1 N HCL at 37° C.) where after 2 hours no more than 45% (by weight) of the racemic tolperisone is released. Alternatively, the composition may exhibit an in vitro dissolution profile (measured using the USP Basket Method at 75 rpm in 1,000 ml 0.1 N HCL at 37° C.) where after 2 hours no more than 55% (by weight) of the racemic tolperisone is released.

As a further alternative, the controlled release pharmaceutical composition may have racemic tolperisone in the amount of 201-500 mg, or pharmaceutically acceptable salts thereof, wherein the composition exhibits an in vitro dissolution profile (measured using the USP Basket Method at 75 rpm in 1,000 ml 0.1 N HCL at 37° C.) where after 2 hours no more than 20% (by weight) of the racemic mixture is released. The controlled release pharmaceutical composition may exhibit an in vitro dissolution profile (measured using the USP Basket Method at 75 rpm in 1,000 ml 0.1 N HCL at 37° C.) where after 2 hours no more than 30% (by weight) of the racemic tolperisone is released. The controlled release pharmaceutical composition may exhibit an in vitro dissolution profile (measured using the USP Basket Method at 75 rpm in 1,000 ml 0.1 N HCL at 37° C.) where after 4 hours no more than 60% (by weight) of the racemic tolperisone has been released.

The present invention further involves a method of treating a chronic disease, benefiting from administration of a muscle relaxant, comprising the daily administration of any of the foregoing discussed controlled release pharmaceutical compositions. Examples of such chronic diseases include multiple sclerosis, fibromyalgia, Parkinson's disease, climacteric symptoms, spasticity resulting from a stroke, spasticity resulting from neurological diseases, cervical syndrome, lumbago, cervico-brachial syndrome, osteoporosis, arthritis, rheumatic diseases such as soft tissue rheumatism and chronic polyarthritis.

The present invention still further involves a controlled release pharmaceutical composition for oral administration to a subject of tolperisone having a core including about 125-175 mg of racemic tolperisone, or pharmaceutically acceptable salts thereof, and a controlled release agent comprising a homogeneous mixture of about 9-12 mg of Eudragit S, about 1.5-2.25 mg Eudragit RS and about 9-12 mg Eudragit L; and a controlled release coating comprising about 1-4 mg Eudragit L associated with the core to provide for controlled release of the racemic tolperisone upon such oral administration resulting in stereoselective disposition of tolperisone enantiomers in the blood plasma of the subject. Preferred embodiments include the controlled release table wherein the controlled release agent comprises a homogeneous mixture of about 10.5 mg Eudragit S, about 1.88 mg Eudragit RS and about 105 mg Eudragit L and the controlled release coating comprises about 2 mg Eudragit L.

Alternatively, a controlled release pharmaceutical composition for oral administration to a subject of tolperisone may have a core including about 300 mg of racemic tolperisone, or pharmaceutically acceptable salts thereof, and a controlled release agent comprising a homogeneous mixture of about 2.5-5 mg Eudragit RS, about 20-22 mg Eudragit L and about 20-22 mg Eudragit S; and a controlled release coating comprising about 4-10 mg Eudragit RS associated with the core to provide for controlled release of the racemic tolperisone upon such oral administration resulting in stereoselective disposition of tolperisone enantiomers in the blood plasma of the subject. Preferred embodiments include the controlled release pharmaceutical composition wherein the controlled release agent comprises about 3.75 mg Eudragit RS, about 21 mg Eudragit L and about 21 mg Eudragit S and the controlled release coating comprises about 4.5 mg of Eudragit RS.

Finally such controlled release of the present application may be effected by racemic tolperisone in a pharmaceutical carrier having a mixture of hydrophilic polymers selected from the group consisting of anionic polymers and cationic polymers and derivatives thereof and combinations thereof dispersed in a hydrophobic matrix. The hydrophilic polymer may be Eudragit S anionic copolymer of methacrylic acid and methacrylic acid methyl ester, Eudragit E cationic copolymer of dimethylaminoethyl methacrylate and neutral methacrylic acid esters, Eudragit RL copolymer of methacrylic acids, Eudragit RS copolymer of methacrylic acids, methacrylic acid polymer, hydroxyethyl methacrylic acid polymer and hydroxymethyl methacrylic acid polymer. The hydrophobic component may be glyceryl dibehenate, glyceryl monostearate, mixtures of glyceryl monostearate and glyceryl monopal reitate, glycerylmonooleate, mixtures of mono, di and tri-glycerides, glycerylmonolaurate, paraffin, white wax, long chain carboxylic acids, long chain carboxylic acid esters and long chain carboxylic acid alcohols.

Such controlled release of the present application may also be effected by an effective amount of racemic tolperisone, a hydrophobic material, and a water sensitive material. The water sensitive material is a hydrophilic polymer which may be Eudragit S anionic copolymer of methacrylic acid and methacrylic acid methyl ester, Eudragit E cationic copolymer of dimethylaminoethyl methacrylate and neutral methacrylic acid esters, Eudragit RL copolymer of methacrylic acids, Eudragit RS copolymer of methacrylic acids, methacrylic acid polymer, hydroxyethyl methacrylic acid polymer and hydroxymethyl methacrylic acid polymer. The hydrophobic material may be glyceryl dibehenate, glyceryl monostearate, mixtures of glyceryl monostearate and glyceryl monopal reitate, glycerylmonooleate, mixtures of mono, di and tri-glycerides, glycerylmonolaurate, paraffin, white wax, long chain carboxylic acids, long chain carboxylic acid esters and long chain carboxylic acid alcohols.

Finally, other means of obtaining controlled release are known in the art. An example is that of U.S. Pat. No. 6,638,534 (Ishibashi et al.) issued Oct. 28, 2003 which is incorporated herein by reference thereto for examples to obtain controlled release.

### Preferred Embodiments

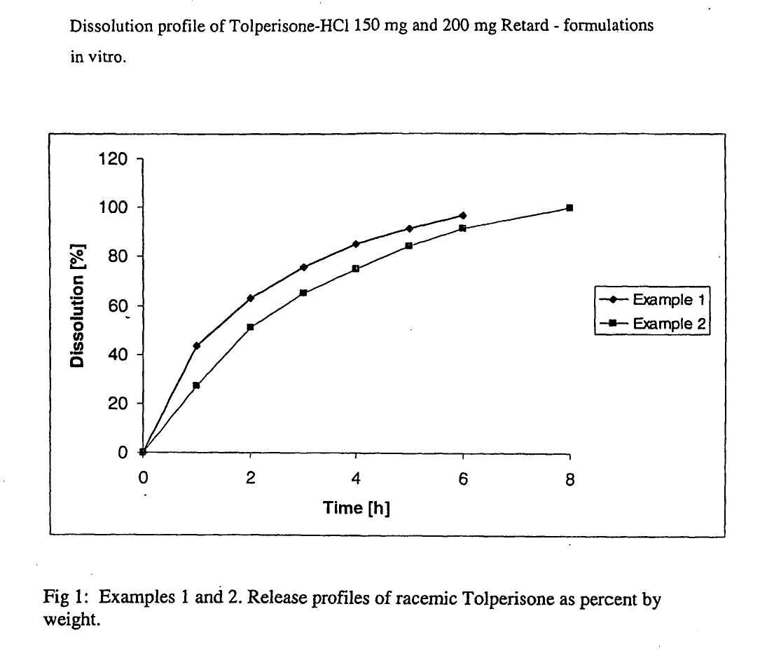

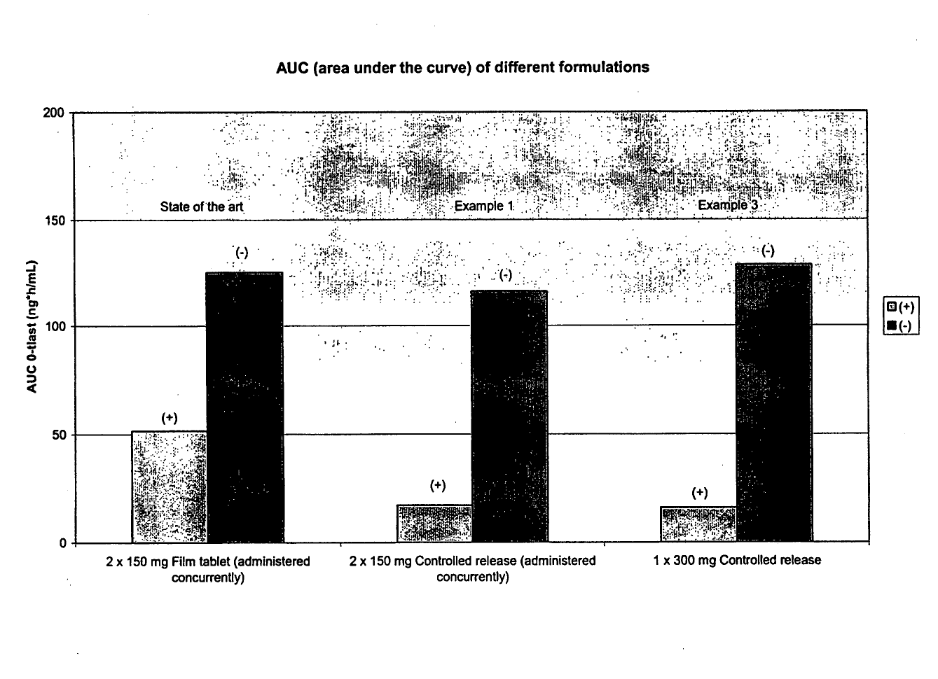

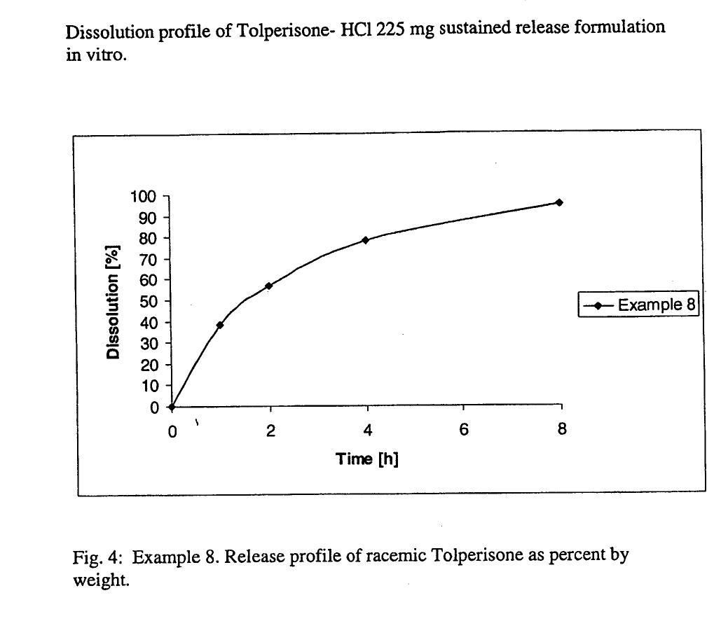

The invention is now explained in detail with reference to exemplary embodiments and with reference to FIGS. 1, 2, 4 and 5 which show the release profiles of preparations according to the examples, and with reference to FIG. 3 relating to the in-vivo fraction of S(+) or R(−)tolperisone.

### EXAMPLE 1

Racemic tolperisone hydrochloride is granulated with a solution consisting of Eudragit RS in butanone in a nixer. Eudragit S and Eudragit L are then mixed in homogeneously, the mixture is dried and sieved. The sieved granular material is then mixed with tabletting excipients and tabletted forming a core. Tablets having a diameter of 8 mm and a weight of 190 mg are pressed, forming a core.

The tablets are then coated with a film material consisting of Eudragit L, colouring agents and other excipients which are dissolved in butanol.

| Ingredient | Amount (mg's) |
|---|---|
| Tolperisone hydrochloride | 150.00 |
| Eudragit RS | 1.88 |
| Eudragit L (core) | 10.50 |
| Eudragit L (coating) | 3.74 |
| Eudragit S | 10.50 |
| Aerosil | 1.80 |
| Stearic acid | 1.80 |
| Glycerol dibehenate | 7.50 |
| Iron oxide colouring agent | 0.08 |
| Titanium dioxide | 4.08 |
| Talc | 6.03 |
| Polyethylene glycol | 1.02 |
| Dimethylpolysiloxane | 0.05 |

It can be seen from FIG. 1 that the preparation according to Example 1 shows a relatively rapid release of active substance, namely approximately 60% in two hours and approximately 85% in four hours. All percentages in this and following examples and the FIGS. Refer to percent by weight dissolved. All amounts of ingredients are stated in mg's in the examples following. After stress storage at 40° C. and 75% humidity, no degradation of the active substance is observed over a period of three months. All tested by-products are below the limit value of <0.2%.

### EXAMPLE 2

In this example the manufacture and composition of a 200 mg racernic tolperisone hydrochloride formulation with average release rate are described. For the manufacture, tolperisone hydrochloride is granulated with a solution. consisting of Eudragit RS in butanone. Eudragit S and Eudragit L are then mixed in homogeneously. The mixture is dried and sieved. After the required tabletting excipients have been homogeneously mixed in, tablets having a diameter of 9 mm and a weight of 250 mg are pressed. These tablets are then film-coated with a solution consisting of Eudragit L, colouring agent and other excipients which are dissolved in butanol.

| Ingredient | Amount (mg's) |
|---|---|
| Tolperisone hydrochloride | 200.00 |
| Eudragit RS | 2.50 |
| Eudragit L | 16.60 |
| Eudragit S | 12.85 |
| Aerosil | 2.40 |
| Stearic acid | 2.40 |
| Glycerol dibehenate | 2.40 |
| Iron oxide colouring agent | 0.08 |
| Titanium dioxide | 4.08 |
| Talc | 10.02 |
| Polyethylene glycol | 1.02 |
| Dimethylpolysiloxane | 0.05 |

The tolperisone-200 mg formulation according to the example shows a release of active substance of approximately 50% in 2 hours and approximately 80% in 5 hours. As can be seen from FIG. 1, this is a comparatively moderate release rate.

### EXAMPLE 3

This example describes the manufacture of a 300 mg racemic tolperisone formulation with constant long-term retardation. Manufacture takes place in a high-speed mixer. Tolperisone is granulated with a granulating solution of Eudragit RS dissolved in butanone. Eudragit L and Eudragit S are then added and dried after homogeneous mixing. The granular material obtained is then mixed homogeneously with tabletting excipients and then pressed into tablets having a diameter of 10 mm and a weight of 380 mg, forming a core. The tablets are film-coated using a solution of Eudragit RS, colouring agent and other excipients in butanone.

| Ingredient | Amount (mg's) |
|---|---|
| Tolperisone hydrochloride | 300.00 |
| Eudragit RS (core) | 3.75 |
| Eudragit RS (coating) | 7.85 |
| Eudragit L | 21.00 |
| Eudragit S | 21.00 |
| Aerosil | 3.60 |
| Stearic acid | 3.60 |
| Glycerol dibehenate | 15.00 |
| Iron oxide colouring agent | 1.26 |
| Titanium dioxide | 6.28 |
| Talc | 14.14 |
| Dimethylpolysiloxane | 0.07 |
| Magnesium stearate | 0.50 |

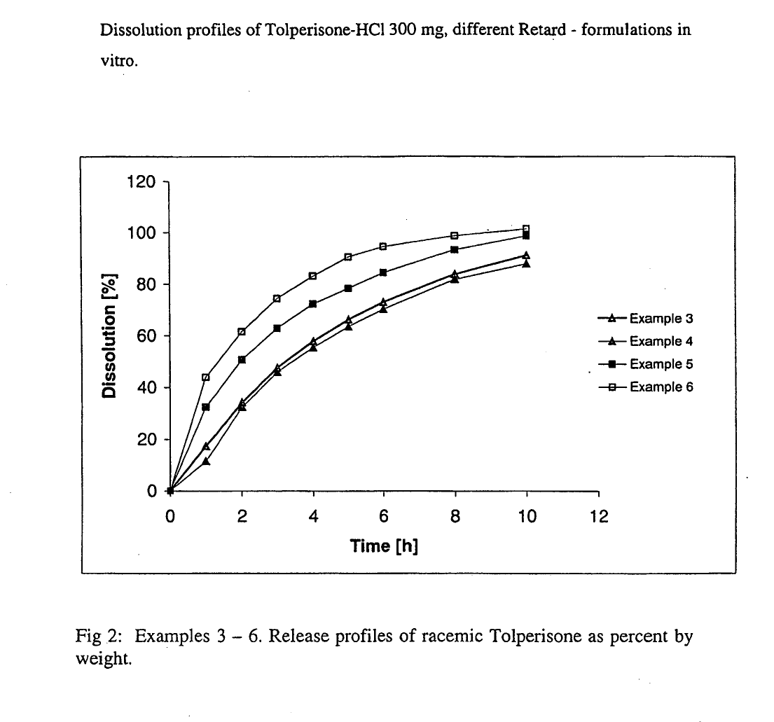

As can be seen from FIG. 2, in the formulation according to the example, the release of active substance is significantly delayed. This means that 50% of the active substance is released after approximately 3 hours and 80% after approximately 7.5 hours. The stability stress test at 40° C. and 75% humidity over 3 months shows tolperisone in a stable form and a fraction of degradation products of <0.02%.

### EXAMPLE 4

This example describes a racemic tolperisone hydrochloride formulation with 300 mg active substance and a very strongly retarded release profile. Manufacture takes place by forming a paste of tolperisone in a pharmaceutical mixer whilst adding a solution consisting of Eudragit RS dissolved in acetone and isopropanol, with Eudragit S and Eudragit L then being mixed in homogeneously. The premixed mass obtained is then dried and sieved. After adding tabletting excipients, tablets are pressed. These tablets are coated with a film consisting of Eudragit RS and colouring agent as well as further pharmaceutical excipients.

| Ingredient | Amount (mg's) |
|---|---|
| Tolperisone hydrochloride | 300.00 |
| Eudragit RS | 27.60 |
| Eudragit L | 21.00 |
| Eudragit S | 21.00 |
| Aerosil | 3.60 |
| Glycerol dibehenate | 18.00 |
| Talc | 22.00 |
| Magnesium stearate | 3.60 |
| Triethyl citrate | 7.50 |

As can be seen from FIG. 2, the formulation according to the example shows a very uniform release of active substance over a long time. That is, 50% of the active substance is released in approximately 3 hours, 80% of the active substance is released in approximately 8 hours. A 100% release of active substance is expected in approximately 12 hours.

### EXAMPLE 5

Example 5 shows a racemic tolperisone formulation with 300 mg of active substance and moderate release rate. The tablet core and the film are manufactured as in Example 4. However, significantly less material is applied.

| Ingredient | Amount (mg's) |
|---|---|
| Tolperisone hydrochloride | 300.00 |
| Eudragit RS | 18.10 |
| Eudragit L | 21.00 |
| Eudragit S | 21.00 |
| Aerosil | 3.60 |
| Glycerol dibehenate | 18.00 |
| Talc | 18.00 |
| Magnesium stearate | 3.60 |
| Triethyl citrate | 4.50 |

FIG. 2 shows a release of active substance of 50% in approximately 2 hours and 80% release after approximately 5.5 hours. The steepness of the curve shows a somewhat faster surge at the beginning of the release and a flattening towards the end of the release of active substance.

### EXAMPLE 6

Example 6 shows a 300 mg racemic tolperisone formulation with slightly delayed release. The tablet core is manufactured as in Example 4. Significantly less film material is used compared with Examples 4 and 5.

| Ingredient | Amount (mg's) |
|---|---|
| Tolperisone hydrochloride | 300.00 |
| Eudragit RS | 8.25 |
| Eudragit L | 21.00 |
| Eudragit S | 21.00 |
| Aerosil | 3.70 |
| Glycerol dibehenate | 18.00 |
| Talc | 14.00 |
| Magnesium stearate | 3.60 |
| Triethyl citrate | 1.50 |

FIG. 2 shows a very rapid release of active substance in the formulation according to the example. Thus, 50% of the active substance is already released after 1.3 hours, and 80% of the active substance after approximately 3.5 hours.

### EXAMPLE 7

Example 7 describes a 150 mg racemic tolperisone-containing formulation with delayed release which additionally has a gastric-juice resistant coating. For its manufacture tolperisone hydrochloride is granulated with Eudragit solution and then dried. The sieved granular material is mixed with tabletting excipients and tabletted. Tablets having a diameter of 8 mm and a weight of 196 mg are pressed. The tablets are coated with a gastric-juice resistant film.

| Ingredient | Amount (mg's) |
|---|---|
| Tolperisone hydrochloride | 150.00 |
| Eudragit RS | 1.88 |
| Eudragit S | 10.20 |
| Triethyl citrate | 0.69 |
| Aerosil | 1.80 |
| Stearic acid | 1.80 |
| Glycerol dibehenate | 7.70 |
| Titanium dioxide | 6.02 |
| Eudragit L | 14.24 |
| Polyethylene glycol | 1.52 |
| Dimethyl polysiloxane | 0.15 |

The film-coated tablets according to the example show none or extremely little release of active substance in gastric juice over a period of 1-2 hours. After buffering to pH 6.8 a somewhat slowed release of active substance takes place.

### EXAMPLE 8

Example 8 describes a racemic tolperisone-containing formulation containing 225 mg tolperisone. Manufacturing takes place by granulating tolperisone with a solution consisting of Eudragit S and Eudragit RS dissolved in butanon and isopropanol. Additional granulation takes place by using a solution of polyvinylpyrrolidone and citric acid in butanone. After sieving, the granulate is homogeniously mixed in a drum blender together with Aerosil, glycerol dibehenate, Eudragit L, stearic.acid and talc. Tablets having a diameter of 9 mm are compressed by using a rotating table compression machine, forming a core. The tablets are coated with a solution consisting of Eudragit L, coloring agents and other excipients which are dissolved in butanol.

| Ingredient | Amount (mg's) |
|---|---|
| Tolperisone hydrochloride | 225.00 |
| Eudragit RS | 2.81 |
| Eudragit L | 19.79 |
| Eudragit S | 15.75 |
| Aerosil | 2.70 |
| Stearic acid | 2.70 |
| Glycerol dibehenate | 11.25 |
| Iron dioxide coloring agent | 0.08 |
| Titanium dioxide | 4.42 |
| Polyetehylene glycol | 1.10 |
| Polysiloxane | 0.05 |

It can be seen from FIG. 3 that the preparation according to Example 8 shows a constant delayed release of tolperisone, namely approximately 80% in 5 hours which is a moderate release rate.

### EXAMPLE 9

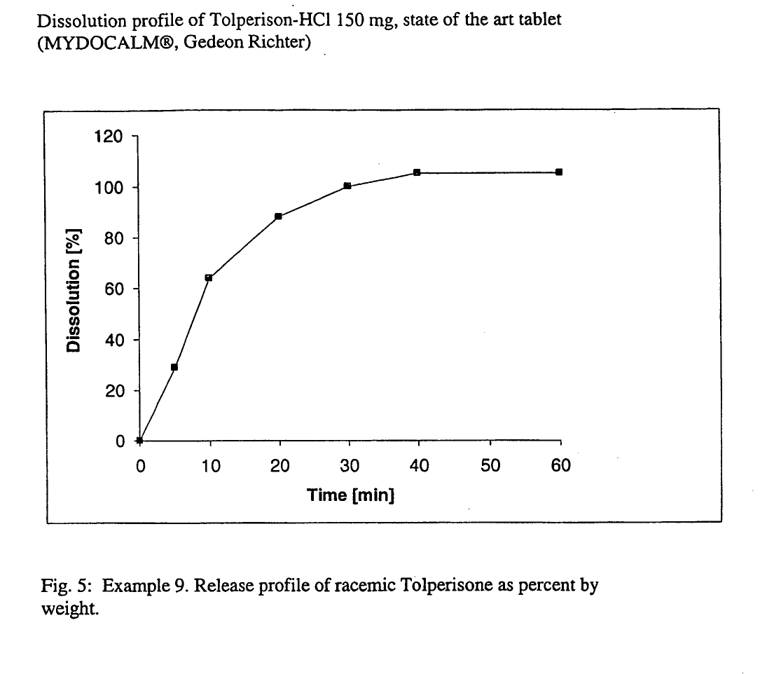

Example 9 describes the preparation which is available commercially as Mydocalm® from Gedeon Richter Ltd, Budapest (Hungary). This preparation was used to create the dissolution profile in FIG. 5 and the (AUC) data in FIG. 3. For FIG. 3 two 150 mg film tablets were administered concurrently. The state of the art preparation contains 150 mg racemic tolperisone HCl; along with the following excipients which bring the tablet to a total weight of 460 mg: citric acid monohydrate; colloidal anhydrous silica; stearic acid; talcum; microcrystalline cellulose; corn starch; lactose monohydrate; Black Iron Oxide; Yellow Iron Oxide; Red Iron Oxide; titanium dioxide; Macrogol 6000; hydroxypropyl methylcellulose 2910.

As a result of the controllable release of active substance by the tolperisone-containing pharmaceutical preparation according to the invention, the in-vivo inversion known in the art can be controlled in the direction of the desired R(−)tolperisone which is effective in muscle-relaxing therapy, thus achieving stereoselective disposition of the enantiomers. In this case, the blood plasma level of patients who were treated with conventional film-coated tablets (the state of the art tablet exemplified by Example 9) shows a larger AUC (Area under the curve) for the S(+)tolperisone which is undesirable in muscle-relaxing therapy.

However, if film-coated tablets such as those manufactured in accordance with Example 1 are administered, the fraction of S(+)tolperisone after in-vivo inversion is thus reduced further in contrast to the in-vivo inversion with known film-coated tablets, thereby achieving stereoselective disposition of the enantiomers.

The same effect was shown using preparations according to the invention such as those of Example 3, wherein the fraction of desired R(−)tolperisone could be additionally increased as a result of the particularly slow and specific release of active substance.

It is possible that plasma proteins preferentially bind to the R(−)tolperisone rather than the S(+)tolperisone and this protects the R(−)tolperisone from first pass degradation or in vivo inversion. Noncontrolled release might exhaust or overwhelm this effect.

Thus, not only the active substance release profile can be specifically adjusted using the tolperisone-containing pharmaceutical preparation according to the invention but at the same time an optimal usage of the in-vivo inversion of enantiomerically pure tolperisone can be achieved in favour of the R(−)enantiomer required for the muscle-relaxing therapy. Accordingly, the tolperisone-containing pharmaceutical formulation according to the invention is used in muscle-relaxing therapy and in the treatment of muscle spasms of various etiology which are triggered by degenerative changes to the spine such as the cervical syndrome, lumbago, cervico-brachial syndrome and similar. However, areas of application are also found in the treatment of osteoporosis as well as arthritis of the knee and/or hip joints and in rheumatic diseases such as soft-tissue rheumatism or chronic polyarthritis. Another area of usage is in the area of treatment of fibromyalgia and in supportive therapy following work and/or sports injuries. The tolperisone-containing pharmaceutical preparation according to the invention is furthermore used in the treatment of spasticity as a result of neurological diseases. Suspensions of tolperisone granules are used with particular advantage if these are administered to children with corresponding flavour enhancers.

However, the tolperisone-containing pharmaceutical preparations according to the invention are also used in the rehabilitation treatment of strokes and in the treatment of multiple sclerosis, Parkinson's disease and climacteric symptoms.

The tolperisone-containing pharmaceutical preparations according to the invention are capable of producing long-lasting uniform levels of action. It can be deduced from recent clinical test reports that tolperisone, especially in high doses, is capable of influencing the pain memory. Under these conditions, tolperisone can also be used successfully to treat diabetic neuropathy, post-herpetic neuralgia and arthritis in Lyme disease (borreliosis).

With reference to the release profiles according to FIGS. 1, 2 and 4, it can be shown that the tolperisone-containing pharmaceutical preparations according to the invention of exemplary embodiments 1 and 6 show a relatively rapid release of active substance whereas the preparations according to Examples 2 and 5 show a moderate release of active substance and those according to Examples 3 and 4 yield a slow but very uniform release of the active substance. Since tolperisone is metabolised at different rates in the human body (which resulted in the classification of four different genotypes with reference to the enzyme CYP2D6 studied in this context, namely the “poor metabolizer”, the “ultrafast metabolizer”, the “extensive metabolizer” and the “intermediate metabolizer”), as a result of its controllable release of active substance, the tolperisone-containing pharmaceutical preparation according to the invention can be matched to the particular genotype according to the rate of release. Thus, the tolperisone-containing pharmaceutical preparations manufactured according to Examples 1 and 6 can be administered to the so-called “ultrafast metabolizer” as a result of their relatively rapid release of active substance, with the formulations according to Examples 2 and 5 being administered to the “extensive” or also to the “intermediate metabolizer” since these respond to tolperisone-containing pharmaceutical preparations with a moderate release of active substance.

In the case of the genotype of the “poor metabolizer” who must be treated with the active substance tolperisone over a relatively long period in order to produce sufficient saturation of the active substance in the blood level, however it is possible to administer the pharmaceutical preparations according to Examples 3 and 4.

The delayed release of tolperisone which is achieved with the pharmaceutical preparation according to the invention can be explained insofar as the active substance tolperisone is predominantly embedded in a polymer matrix which is pharmaceutically compatible and which during the metabolisation of tolperisone allows a delayed but specific release of tolperisone as a result of the embedding in the matrix material. This release can be additionally supported by the fact that in the case of tablets, the tablet cores are additionally surrounded by a coating which delays the release of the active substance. The materials used for this coating advantageously consist of pharmaceutically compatible polymers which likewise bring about a slowed but at the same time controllable release as a result of their matrix structure. A uniform saturation of tolperisone in the blood plasma level is thereby achieved so that undesirable, so-called “overshooting peaks” in the blood plasma level accordingly can be avoided. This yields an advantageous effect in the administration of the tolperisone-containing pharmaceutical formulation according to the invention with regard to the rare “poor metabolizer” and “ultrafast metabolizer” CYP2D6 genotype groups. As a result of the specific release, a reduction in the toxicity risk and thus a reduction in the rate of side effects is obtained for the “poor metabolizer”, whereas in the case of the “ultrafast metabolizer” a more uniform and therefore improved level of action can be achieved over a longer time compared with conventional film-coated tablets. As a result of the controllable and therefore uniform release of active substance, the aforesaid genotypes are supplied with the active substance tolperisone over a longer time so that the blood plasma level is sufficiently saturated with tolperisone.

In summary, the tolperisone-containing pharmaceutical preparation according to the invention allows a specific and controllable dosing without free active substance insofar as the active principle tolperisone is embedded in a suitable pharmaceutical carrier, preferably a polymer matrix. As a result, by selecting the materials for the matrix or coating of the tablet or granules, a release of active substance matched to the special genotype can be achieved. At the same time, as a result of the very uniform and persistent release of tolperisone, the known in-vivo inversion of enantomerically pure tolperisone can be adjusted in favour of the R(−)tolperisone relevant in muscle-relaxing therapy.

1. A controlled release pharmaceutical composition for oral administration of tolperisone to a subject comprising an amount of enantiomeric mixture of tolperisone, or pharmaceutically acceptable salts thereof, and a controlled release agent to provide for controlled release of the enantiomeric mixture of tolperisone upon such oral administration resulting in stereoselective disposition of tolperisone enantiomers in the blood plasma of the subject wherein the plasma area under the curve (AUC) concentration ratio of R-tolperisone to S-tolperisone is higher than that of a non-controlled release composition containing the same amount of enantiomeric mixture of tolperisone.

2. The controlled release pharmaceutical composition of claim 1 wherein the pharmaceutical composition further comprises (a) a core which includes (i) the enantiomeric mixture of tolperisone and (ii) the controlled release agent and (b) a controlled release coating associated with the core.

3. The controlled release pharmaceutical composition of claim 1 wherein the enantiomeric mixture of tolperisone is a racemic mixture.

4. The controlled release pharmaceutical composition of claim 3 wherein the amount of racemic mixture in the core is within the range of 100-249 mg.

5. The controlled release pharmaceutical composition of claim 1 wherein the controlled release of the racemic mixture of tolperisone results in no more than 55% by weight at 2 hours (measured using the USP Basket Method at 75 rpm in 1,000 ml 0.1 HCL at 37° C.).

6. The controlled release pharmaceutical composition of claim 4 wherein the controlled release of the racemic mixture of tolperisone results in no more than 45% by weight at 2 hours (measured using the USP Basket Method at 75 rpm in 1,00O mI O-I HCL aI SV0 C).

7. The controlled release pharmaceutical composition of claim 3 wherein the amount of racemic mixture in the core is within the range of 250-500 mg.

8. The controlled release pharmaceutical composition of claim 7 wherein the composition exhibits an in vitro dissolution profile (measured using the USP Basket Method at 75 rpm in 1,000 ml 0.1 N HCL at 37° C.) where after 2 hours no more than 20% (by weight) of the racemic mixture is released.

9. The controlled release pharmaceutical composition of claim 7 wherein the composition exhibits an in vitro dissolution profile (measured using the USP Basket Method at 75 rpm in 1,000 ml 0.1 N HCL at 37° C.) where after 2 hours no more than 30% (by weight) of the racemic mixture is released.

10. The controlled release pharmaceutical composition of claim 7 wherein the composition further exhibits an in vitro dissolution profile (measured using the USP Basket Method at 75 rpm in 1,000 ml 0.1 N HCL at 37° C.) where after 4 hours no more than 60% (by weight) of the racemic mixture has been released.

11. The controlled release pharmaceutical composition of claim 1 wherein the controlled release agent is a mixture of anionic and cationic acrylic polymers.

12. The controlled release pharmaceutical composition of claim 11 wherein said mixture of anionic and cationic acrylic polymers is a mixture of Eudragit RS, Eudragit L and Eudragit S.

13. The controlled release pharmaceutical composition of claim 1 wherein the controlled release coating is pH independent.

14. The controlled release pharmaceutical composition of claim 13 wherein the controlled release coating is Eudragit RS.

15. The controlled release pharmaceutical composition of claim 1 wherein the controlled release coating is pH dependent.

16. The controlled release pharmaceutical composition of claim 15 wherein the controlled release coating is Eudragit L.

17. A controlled release pharmaceutical composition for oral administration of tolperisone to a subject comprising an amount of racemic tolperisone, or pharmaceutically acceptable salts thereof, and a controlled release agent to provide for controlled release of the racemic tolperisone upon such oral administration resulting in stereoselective disposition of tolperisone enantiomers in the blood plasma of the subject wherein the plasma area under the curve (AUC) concentration ratio of R-tolperisone to S-tolperisone is 3:1 or higher.

18. The controlled release pharmaceutical composition of claim 17 wherein the pharmaceutical composition further comprises (a) a core which includes (i) the racemic mixture of tolperisone and (ii) the controlled release agent and (b) a controlled release coating associated with the core.

19. The controlled release pharmaceutical composition of claim 17 wherein the plasma area under the curve (AUC) concentration ratio is 4:1 or higher.

20. The controlled release pharmaceutical composition of claim 17 wherein the amount of racemic tolperisone is within the range of 100-249 mg.

21. The controlled release pharmaceutical composition of claim 20 wherein the controlled release of the racemic tolperisone results in no more than 55% by weight at 2 hours (measured using the USP Basket Method at 75 rpm in 1,000 ml 0.1 HCL at 37° C.).

22. The controlled release pharmaceutical composition of claim 20 wherein the controlled release of the racemic tolperisone results in no more than 45% by weight at 2 hours (measured using the USP Basket Method at 75 rpm in 1,000 ml 0.1 HCL at 37° C.).

23. The controlled release pharmaceutical composition of claim 17 wherein the amount of racemic tolperisone is within the range of 250-500 mg.

24. The controlled release pharmaceutical composition of claim 23 wherein the composition exhibits an in vitro dissolution profile (measured using the USP Basket Method at 75 rpm in 1,000 ml 0.1 N HCL at 37° C.) where after 2 hours no more than 20% (by weight) of the racemic mixture is released.

25. The controlled release pharmaceutical composition of claim 23 wherein the composition exhibits an in vitro dissolution profile (measured using the USP Basket Method at 75 rpm in 1,000 ml 0.1 N HCL at 37° C.) where after 2 hours no more than 30% (by weight) of the racemic tolperisone is released.

26. The controlled release pharmaceutical composition of claim 23 wherein the composition further exhibits an in vitro dissolution profile (measured using the USP Basket Method at 75 rpm in 1,000 ml 0.1 N HCL at 37° C.) where after 4 hours no more than 60% (by weight) of the racemic tolperisone has been released.

27. The controlled release pharmaceutical composition of claim 17 wherein the controlled release agent is a mixture of anionic and cationic acrylic polymers.

28. The controlled release pharmaceutical composition of claim 27 wherein said mixture of anionic and cationic acrylic polymers is a mixture of Eudragit RS, Eudragit L and Eudragit S.

29. The controlled release pharmaceutical composition claim 17 wherein the controlled release coating is pH independent.

30. The controlled release pharmaceutical composition of claim 29 wherein the controlled release coating is Eudragit RS.

31. The controlled release pharmaceutical composition of claim 17 wherein the controlled release coating is pH dependent.

32. The controlled release pharmaceutical composition of claim 31 wherein the controlled release coating is Eudragit L.

33. A method of oral administration of tolperisone to a subject comprising: oral administration by controlled release of a dose of an amount of racemic tolperisone in the range of 100-500 mg to provide a stereoselective disposition of tolperisone enantiomers in the blood plasma of the subject.

34. The method of claim 33 wherein the amount of racemic tolperisone is in the range of about 300 mg.

35. The method of claim 33 wherein the amount of racemic tolperisone is in the range of about 150 mg.

36. The method of claim 33 wherein the amount of racemic tolperisone is in the range of 250-350 mg and the stereoselective disposition of tolperisone enantiomers in the blood plasma of the subject has a plasma area under the curve (AUC) concentration of R-tolperisone of 100 ng*h/ml or higher and such concentration for S-tolperisone is 25 ng*h/ml or lower.

37. A controlled release pharmaceutical composition comprising racemic tolperisone in the amount of 100-200 mg, or pharmaceutically acceptable salts thereof, wherein the composition exhibits an in vitro dissolution profile (measured using the USP Basket Method at 75 rpm in 1,000 ml 0.1 N HCL at 37° C.) where after 2 hours no more than 45% (by weight) of the racemic tolperisone is released.

38. The controlled release pharmaceutical composition of claim 37 wherein the composition exhibits an in vitro dissolution profile (measured using the USP Basket Method at 75 rpm in 1,000 ml 0.1 N HCL at 37° C.) where after 2 hours no more than 55% (by weight) of the racemic tolperisone is released.

39. The controlled release pharmaceutical composition of claim 37 wherein the composition further comprises: a core including the racemic tolperisone, or pharmaceutically acceptable salts thereof, and a controlled release agent; and a controlled release coating associated with the core.

40. The controlled release pharmaceutical composition of claim 39 wherein the composition provides upon oral administration to a subject for controlled release of the racemic tolperisone resulting in stereoselective disposition of tolperisone enantiomers in the blood plasma of the subject.

41. A controlled release pharmaceutical composition comprising racemic tolperisone in the amount of 201-500 mg, or pharmaceutically acceptable salts thereof, wherein the composition exhibits an in vitro dissolution profile (measured using the USP Basket Method at 75 rpm in 1,000 ml 0.1 N HCL at 37° C.) where after 2 hours no more than 20% (by weight) of the racemic mixture is released.

42. The controlled release pharmaceutical composition of claim 41 wherein the composition exhibits an in vitro dissolution profile (measured using the USP Basket Method at 75 rpm in 1,000 ml 0.1 N HCL at 37° C.) where after 2 hours no more than 30% (by weight) of the racemic tolperisone is released.

43. The controlled release pharmaceutical composition of claim 42 wherein the composition further exhibits an in vitro dissolution profile (measured using the USP Basket Method at 75 rpm in 1,000 ml 0.1 N HCL at 37° C.) where after 4 hours no more than 60% (by weight) of the racemic tolperisone has been released.

44. The controlled release pharmaceutical composition of claim 41 wherein the composition further comprises: a core including the racemic tolperisone, or pharmaceutically acceptable salts thereof, and a controlled release agent; and a controlled release coating associated with the core.

45. The controlled release pharmaceutical composition of claim 44 wherein the composition provides upon oral administration to a subject for controlled release of the racemic tolperisone resulting in stereoselective disposition of tolperisone enantiomers in the blood plasma of the subject.

46. A method of treating a chronic disease, benefiting from administration of a muscle relaxant, comprising administering the pharmaceutical composition of claim 1 to a subject in need thereof.

47. The method of claim 46 wherein the chronic disease is selected from the group consisting of multiple sclerosis, fibromyalgia, Parkinson's disease, climacteric symptoms, spasticity resulting from a stroke, spasticity resulting from neurological diseases, cervical syndrome, lumbago, cervico-brachial syndrome, osteoporosis, arthritis, rheumatic diseases such as soft tissue rheumatism and chronic polyarthritis.

48. The method of claim 47 wherein the chronic disease is multiple sclerosis.

49. The method of claim 47 wherein the chronic disease is spasticity resulting from a stroke.

50. The method of claim 47 wherein the chronic disease is spasticity resulting from neurological diseases.

51. The method of claim 47 wherein the chronic disease is fibromyalgia.

52. The method of claim 47 wherein the chronic disease is Parkinson's disease.

53. The method of claim 47 wherein the chronic disease is climacteric symptoms.

54. The method of claim 47 wherein the chronic disease is cervical syndrome.

55. The method of claim 47 wherein the chronic disease is cervico-brachial syndrome.

56. The method of claim 47 wherein the chronic disease is osteoporosis.

57. The method of claim 47 wherein the chronic disease is arthritis.

58. The method of claim 47 wherein the chronic disease is rheumatic diseases such as soft tissue rheumatism and chronic polyarthritis.

59. A controlled release pharmaceutical composition for oral administration to a subject of tolperisone comprising: a core including about 125-175 mg of racemic tolperisone, or pharmaceutically acceptable salts thereof, and a controlled release agent comprising a homogeneous mixture of about 9-12 mg of Eudragit S, about 1.5-2.25 mg Eudragit RS and about 9-12 mg Eudragit L; and a controlled release coating comprising about 1-4 mg Eudragit L associated with the core to provide for controlled release of the racemic tolperisone upon such oral administration resulting in stereoselective disposition of tolperisone enantiomers in the blood plasma of the subject.

60. The controlled release table of claim 59 wherein the controlled release agent comprises a homogeneous mixture of about 10.5 mg Eudragit S, about 1.88 mg Eudragit RS and about 105 mg Eudragit L and the controlled release coating comprises about 2 mg Eudragit L.

61. A controlled release pharmaceutical composition for oral administration to a subject of tolperisone comprising: a core including about 300 mg of racemic tolperisone, or pharmaceutically acceptable salts thereof, and a controlled release agent comprising a homogeneous mixture of about 2.5-5 mg Eudragit RS, about 20-22 mg Eudragit L and about 20-22 mg Eudragit S; and a controlled release coating comprising about 4-10 mg Eudragit RS associated with the core to provide for controlled release of the racemic tolperisone upon such oral administration resulting in stereoselective disposition of tolperisone enantiomers in the blood plasma of the subject.

62. The controlled release pharmaceutical composition of claim 51 wherein the controlled release agent comprises about 3.75 mg Eudragit RS, about 21 mg Eudragit L and about 21 mg Eudragit S and the controlled release coating comprises about 4.5 mg of Eudragit RS.

63. A controlled release pharmaceutical composition for the oral administration of racemic tolperisone to a subject in need thereof comprising racemic toleprisone in a pharmaceutical carrier comprising a mixture of hydrophilic polymers selected from the group consisting of anionic polymers and cationic polymers and derivatives thereof and combinations thereof dispersed in a hydrophobic matrix.

64. The composition of claim 63 wherein the hydrophilic polymer is selected from the group consisting of Eudragit S anionic copolymer of methacrylic acid and methacrylic acid methyl ester, Eudragit E cationic copolymer of dimethylaminoethyl methacrylate and neutral methacrylic acid esters, Eudragit RL copolymer of methacrylic acids, Eudragit RS copolymer of methacrylic acids, methacrylic acid polymer, hydroxyethyl methacrylic acid polymer and hydroxymethyl methacrylic acid polymer.

65. The composition of claim 63 wherein the hydrophobic component is selected from the group consisting of glyceryl dibehenate, glyceryl monostearate, mixtures of glyceryl monostearate and glyceryl monopal reitate, glycerylmonooleate, mixtures of mono, di and tri-glycerides, glycerylmonolaurate, paraffin, white wax, long chain carboxylic acids, long chain carboxylic acid esters and long chain carboxylic acid alcohols.

66. A controlled release formulation of racemic tolperisone for oral administration comprising: an effective amount of racemic tolperisone, a hydrophobic material, and a water sensitive material.

67. The composition of claim 66 wherein the water sensitive material is a hydrophilic polymer selected from the group consisting of Eudragit S anionic copolymer of methacrylic acid and methacrylic acid methyl ester, Eudragit E cationic copolymer of dimethylaminoethyl methacrylate and neutral methacrylic acid esters, Eudragit RL copolymer of methacrylic acids, Eudragit RS copolymer of methacrylic acids, methacrylic acid polymer, hydroxyethyl methacrylic acid polymer and hydroxymethyl methacrylic acid polymer.

68. The composition of claim 66 wherein the hydrophobic material is selected from the group consisting of glyceryl dibehenate, glyceryl monostearate, mixtures of glyceryl monostearate and glyceryl monopal reitate, glycerylmonooleate, mixtures of mono, di and tri-glycerides, glycerylmonolaurate, paraffin, white wax, long chain carboxylic acids, long chain carboxylic acid esters and long chain carboxylic acid alcohols.

69. The controlled release pharmaceutical composition of claim 1 wherein the enantiomeric mixture of tolperisone has at least 50% by weight the R-tolperisone and no less than 10% by weight the S-tolperisone.

70. A method of treating a chronic disease, benefiting from administration of a muscle relaxant, comprising administering the pharmaceutical composition of claim 1 to a subject in need thereof.

71. A method of treating a chronic disease, benefiting from administration of a muscle relaxant, comprising administering the pharmaceutical composition of claim 17 to a subject in need thereof.

72. A method of treating a chronic disease, benefiting from administration of a muscle relaxant, comprising administering the pharmaceutical composition of claim 37 to a subject in need thereof.

73. A method of treating a chronic disease, benefiting from administration of a muscle relaxant, comprising administering the pharmaceutical composition of claim 41 to a subject in need thereof.

74. A method of treating a chronic disease, benefiting from administration of a muscle relaxant, comprising administering the pharmaceutical composition of claim 59 to a subject in need thereof.

75. A method of treating a chronic disease, benefiting from administration of a muscle relaxant, comprising administering the pharmaceutical composition of claim 61 to a subject in need thereof.

76. A method of treating a chronic disease, benefiting from administration of a muscle relaxant, comprising administering the pharmaceutical composition of claim 63 to a subject in need thereof.

77. A method of treating a chronic disease, benefiting from administration of a muscle relaxant, comprising administering the pharmaceutical composition of claim 66 to a subject in need thereof.
